(19) United States
(12) Patent Application Publication
(10) Pub. No.: US 2009/0239812 A1
(43) Pub. Date: Sep. 24, 2009

(54) USE OF C7 SUGARS IN PREVENTION AND TREATMENT OF MYCOSES

(75) Inventor: Philippe Msika, Versailles (FR)

Correspondence Address: FOLEY AND LARDNER LLP;SUITE 500, 3000 K STREET NW, WASHINGTON, DC, 20007, US

(73) Assignee: Laboratoires Expanscience

(21) Appl. No.: 12/310,450

(22) PCT Filed: Aug. 31, 2007
(86) PCT No.: PCT/EP2007/059136

§ 371 Date: Feb. 25, 2009

(30) Foreign Application Priority Data
Aug. 31, 2006 (FR) 0607651

(51) Int. Cl. A61K 31/7004; A61P 31/10

(52) U.S. Cl. 514/23

(57) ABSTRACT

The object of the invention is use of at least one C7 sugar to make a composition intended for prevention and/or treatment of mycoses selected from among the group constituted of candidoses and pityrosporoses. The C7 sugars are preferably a water soluble extract of avocado sugars.

The present invention relates to the use of at least one C7 sugar to prevent and/or fight the proliferation of yeasts present on the skin, skin appendages and/or mucosae.

Classified in the kingdom Fungi, fungi are eukaryotic organisms that are heterotrophic with respect to carbon, feed by absorption and reproduce by sporulation. Microscopic fungi are subdivided in molds, dermatophytes and yeasts (unicellular organisms). Some microscopic fungi can cause in man parasitic diseases of the skin, skin appendages and viscera.

Mycoses are parasitoses caused by the proliferation of parasitic, microscopic fungi in the body. The most common mycoses include candidiases and pityrosporoses, which are caused by the proliferation of yeast on the skin.

Candidiases are diseases caused by yeasts of the genus Candida. There are two forms of candidiases according to their development: acute mycoses (most common), caused by local factors, and chronic mycoses (rare), whose treatment is more difficult and which are sometimes caused by an immune system deficiency.

Cutaneous candidiases (affecting the skin) are located primarily in body folds and are supported by maceration (excess moisture or insufficient drying of the skin). Cutaneous candidiases result in the presence of an intertrigo, i.e., a lesion that begins at the bottom of cutaneous folds, becomes erythematous (red in color) and which finishes by oozing and causing pruritus (itching). Cutaneous candidiases can also occur at the base of the nails, causing superficial paronychia.

Oral candidiases cause thrush, which is characterized by whitish and bright red areas on the inside of the cheeks. Oral candidiases appear primarily in individuals suffering from an absence of saliva (aptyalism). Oral candidiases are facilitated in immunosuppressed individuals (those with low immune defenses), as well as those with HIV (AIDS) and those with dental appliances.

Genital candidiases are the source in women of vulvovaginitis with vaginal discharge and in men balanitis (inflammation of the prepuce). Genital candidiases in males are the source of pruritus and rather frequent urethral discharge. Other candidiases are aggravations of gluteal erythema in infants due to maceration and an increase in pH which becomes alkaline.

Pityrosporoses are very common infections of the skin caused by yeasts of the genus Pityrosporum.

There are various types of pityrosporoses, most notably:
pityriasis versicolor, a dermatosis characterized by the appearance of diffuse redness (erythema) associated with very mild desquamation (mild shedding of the outer layers of the skin) and caused by germination of a yeast (Pityrosporum orbiculare, also called Microsporum furfur or Malassezia furfur),
pityriasis of the scalp, characterized by the presence of dandruff (scales of variable size) sometimes associated with itching,
Brocq's dermatosis, characterized by the presence of very small red patches whose center is paler, associated with mild shedding of skin, sometimes with itching, and located on the chest,
seborrheic dermatitis, characterized by the presence of small reddish or sometimes yellow-orange plaques covered with scales (small pieces of skin) of liquid appearance (greasy), without itching. These dermatological lesions occur high on the face at the edge of the scalp, around the eyebrows and on the ala of the nose,
pityriosporum folliculitis, located on the back and characterized by small papules accompanied by mild itching.

Treatments to prevent or cure these mycoses, or their symptoms, are known but the person skilled in the art continue to search for alternative treatments.

Thus, in an unexpected and surprising way, the inventors have discovered that C7 sugars, in particular those extracted from avocado, can cause the physical agglomeration or aggregation of these fungi, thus preventing them from penetrating into cells and consequently inhibiting their pathogenicity.

D-mannoheptulose, the first ketoheptose identified in 1916 by La Forge, of general formula (I)

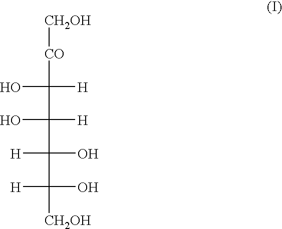

is found in certain plants, in particular alfalfa (Medicago sativa L.), avocado, fig (Ficus officinalis L.), showy stonecrop (Sedum spectabile Bor.) and primrose (Primula officinalis Jacq.). However, the highest content of D-mannoheptulose is found in avocado. D-mannoheptulose has already been used in therapeutic applications. For example, patent application WO95/03809 describes the use of D-mannoheptulose as a glucokinase inhibiter to inhibit development of tumor cells and application US2003/0092669 describes an oral food supplement containing D-mannoheptulose that decreases insulin level and thus enables weight loss.

Perseitol, the polyol form of D-mannoheptulose, of general formula (II)

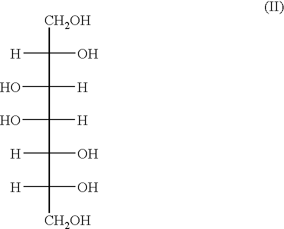

is also found in avocado, in particular in the fruit or pit.

According to the publication “Search for pharmacochemical leads from tropical rainforest plants”, Hitotaka Shibuya et al. Pure Appl. Chem., vol. 71, no. 6, pp. 1109-1113, 1999, perseitol, associated with a potassium ion, inhibits incorporation of 3H-leucine in Ehrlich's ascites tumor cells.

The use of these sugars (perseitol and D-mannoheptulose) to stimulate synthesis of human beta-defensins (in particular HBD-2) has already been described (WO 2005/115421).

The invention relates to the use of at least one C7 sugar to manufacture a composition to prevent and/or treat mycoses selected from the group comprised of candidiases and pityrosporoses.

The inventors have noted that when C7 sugars are brought into contact with particular yeasts of the genera Candida and Pityrosporum, these sugars are able to aggregate said yeasts. Once aggregated, said yeasts have a limited ability to move; since they are larger, they can no longer penetrate keratinocytes.

It is assumed that this aggregation is caused by the presence of mannose residues on the surface of these yeasts. Indeed, whereas C7 sugars are able to agglomerate these yeasts, they do not induce the agglomeration of fungi lacking these residues on their surface or of bacteria such as S. aureus, P. aeruginosa and S. pyogenes (which do not have mannose residues on their surface).

The yeasts targeted by the invention thus advantageously have mannose residues on their surface.

Moreover, the inventors have discovered that C7 sugars are able to inhibit yeast pathogenicity.

One way in which yeasts act is to modulate the proinflammatory action of the host to ensure their survival. Thus, the yeast M. furfur is able to decrease production of the pro-inflammatory cytokines that are supposed to fight against its propagation such as IL-1α, IL-6 and TNF-α and increase production of TGF-β1 (an immunosuppressive and anti-inflammatory cytokine) by keratinocytes. Suppression of these interleukins enables yeasts to survive in the host without them generating an anti-inflammatory response.

However, it was noted that when keratinocytes are pretreated with C7 sugars, these keratinocytes regain their capacity to respond with a proper proinflammatory reaction with respect to yeasts. C7 sugars are thus able to inhibit yeast pathogenicity and to restore the defensive capacities of keratinocytes which again become intolerant to yeasts.

Within the scope of the present invention, the targeted candidiases are in particular those selected from the group comprised of candidal intertrigo, oral-digestive candidiasis, adult or infantile genital candidiasis, candidal onychitis and perionychitis, chronic mucocutaneous candidiasis and candidal folliculitis.

Candidiasis is caused by yeasts of the genus Candida, most notably C. albicans. Under certain physiological (pregnancy), pathological (diabetes) or iatrogenic conditions (treatment with hormones, in particular corticoids, with antibiotics or with immunosuppressants), Candida can become pathogenic and cause various cutaneous and mucosal symptoms.

In addition to the general factors cited above, candidal intertrigo is supported by obesity, maceration and poor hygiene.

Oral-digestive candidiasis can reach all segments of the digestive system. Types include perleche, cheilitis and stomatitis (thrush). These candidiases are often marked by erythema.

Genital candidiases are most frequently seen in adults or in infants by the extension of diaper rash. Types include candidal vulvovaginitis, urethritis (common in diabetics), balanitis and balanoposthitis, and infantile candidiasis of the genitals and buttocks, which affects very young infants and is generally known as diaper rash (which can have various causes).

Candidal onychitis and perionychitis are supported by moisture.

Chronic mucocutaneous candidiases are chronic cutaneous and mucosal infections, which occur among patients with immune deficiencies.

Candidal folliculitis is particularly associated with heroin addiction and is a reflection of septicemia.

In a preferred embodiment of the invention, the candidiases are selected from the group comprised of oral-digestive candidiasis, genital candidiasis, infantile candidiasis of the genitals and buttocks, in particular diaper rash, and candidal folliculitis.

Within the scope of the present invention, the pityrosporoses targeted are in particular those selected from the group comprised of pityriasis versicolor, pityriosporum folliculitis, seborrheic dermatitis, acne, rosacea and atopic dermatitis.

By definition, a pellicular state consists of non-inflammatory desquamation of the scalp. Seborrheic dermatitis is characterized by the presence of erythema and an extension of the disease beyond the scalp. Here, Malassezia sp. plays a central role and is responsible for an inflammatory reaction leading to epidermal deterioration.

In a preferred embodiment of the invention, the pityrosporoses are selected from the group comprised of pityriosporum folliculitis, seborrheic dermatitis, acne, rosacea and atopic dermatitis.

In the invention, the mycoses targeted are candidiases (in particular those caused by the proliferation of Candida spp., in particular C. albicans) and pityrosporoses (in particular those caused by the proliferation of Malassezia spp., in particular M. furfur).

Yeasts of the genus Malassezia (Malassezia spp.) are associated with numerous systemic and cutaneous diseases, either as their cause or with which they are associated. Examples of diseases related to Malassezia spp. notably include pityriasis versicolor, seborrheic dermatitis, pellicles, folliculitis, atopic dermatitis, psoriasis, reticulated papillomatosis and seborrheic blepharitis. It has also been shown that Malassezia spp. is associated with deeper infections, such as mastitis, sinusitis, purulent arthritis, malignant external otitis, fungemia, angeitis, peritonitis and meningitis. (Helen Ruth Ashbee, Recent developments in the immunology and biology of Malassezia species, HEMS Immunol. Med. Microbiol. 47 (2006) 14-23).

Thus, the inventors have shown that C7 sugars are able to agglomerate yeasts (which are then too large to penetrate cells) and to restore keratinocyte tolerance to these yeasts. These yeasts lose their capacity to invade and their pathogenicity without the sugars at these concentrations having the capacity to kill or inhibit the growth of the attackers (not a fungicide or fungistat).

Moreover, C7 sugars have a mode of action that is physical and not chemical. Thus, they have long-term effectiveness, contrary to chemical effectors which are likely to induce resistance by the pathogens.

This long-term effect is particularly desirable because it enables the treatment of patients with mycoses that are insensitive to the standard treatments available to date, most notably due to resistance to the chemical effectors used by yeasts.

The inventive C7 sugars are thus useful in the treatment and prevention of mycoses. They can also be associated with other medications to treat diseases (primarily cutaneous diseases) where the proliferation of yeasts can be a symptom without being the cause. Thus, for example, C7 sugars can be used to limit the proliferation of Malassezia spp. in the case of psoriasis.

According to an advantageous embodiment of the invention, C7 sugars are used within the context of a treatment for diaper rash. They can be used alone or in combination with other active ingredients.

It is interesting to note that the physical action of C7 sugars on yeast aggregation does not disturb cutaneous equilibrium, which is particularly fragile and sensitive in babies and infants. Thus, these sugars are particularly suited to treat babies and infants or other patients with fragile cutaneous equilibrium for whom standard treatments must be used parsimoniously since side effects can be severe.

The active ingredients that can be used in combination are zinc oxide, talc, titanium oxide, vitamin B5, polyphenols, antienzyme (urease, lipase, protease) such as soy isoflavone, oleyl acetate and water in oil or in silicone emulsifiers that protect the infant's skin.

The active ingredients that can be used in combination are also procyanidolic oligomers (PCOs) and anthocyanins, such as those described in application EP 953,353.

According to another advantageous embodiment of the invention, C7 sugars are used within the context of a treatment for dandruff or seborrheic dermatitis, preferably seborrheic dermatitis. They can be used alone or in combination with other active ingredients.

The active ingredients that can be used in combination are standard antifungals (ketoconazole, zinc pyrithione, piroctone olamine, etc.), anti-lipases, selenium and lithium salts, tars (coal tar, ichtyol pale, etc.), anti-inflammatories, exfoliants or keratin regulators (AHA, polyhydroxylated acids, esterified or substituted free BHA, etc.).

According to another advantageous embodiment of the invention, C7 sugars are used within the context of a treatment for atopy. They can be used alone or in combination with other active ingredients.

Atopy describes a clinical state of constitutional or hereditary hypersensitivity, predisposing the subject to allergic reactions in the presence of allergens that do not trigger a reaction in normal subjects.

The active ingredients that can be used in combination are local corticoids, local or systemic immunosuppressors, cosmetic emollients, anti-inflammatories, stimulants of lipid biosynthesis (sunflower or avocado furan concentrates), PPAR agonists, stimulants of antimicrobial peptides (leucine, avocado peptides, etc.).

According to another advantageous embodiment of the invention, C7 sugars are used within the context of the treatment or prevention of psoriasis or its symptoms.

Psoriasis is an autoimmune disease characterized by a proliferation of fungi, in particular the yeast Malassezia spp. (Helen Ruth Ashbee, Recent developments in the immunology and biology of Malassezia species, HEMS Immunol. Med. Microbiol. 47 (2006) 14-23). The inventive C7 sugars limit the proliferation of this yeast and thus limit the severity and extent of lesions.

The active ingredients that can be used in combination are cortisone, dermocorticoids, vitamin D derivatives (in particular calcipotriol), retinoids, immunosuppressors, methotrexate, cyclosporine, extracts of Centella asiatica and keratolytics.

According to another advantageous embodiment of the invention, C7 sugars are used within the context of the treatment or prevention of inflammatory acne or comedones. The active ingredients that can be used in combination are local or systemic retinoids, local or systemic antibiotics, local antibacterials, AHA, polyhydroxylated acids, BHA, anti-inflammatories, cutaneous cicatrizers and zinc salts.

According to another advantageous embodiment of the invention, C7 sugars are used within the context of the treatment or prevention of rosacea. The active ingredients that can be used in combination are metronidazole, flavonoids, PCOs, polyphenols, isoflavones, anti-collagenases/elastases/gelatinases and anti-inflammatories.

According to another advantageous embodiment of the invention, C7 sugars are used within the context of the treatment or prevention of dermatitis of the body folds and genitals. The active ingredients that can be used in combination are standard antibacterials or antifungals and drying agents (eosin, potassium permanganate).

According to another advantageous embodiment of the invention, C7 sugars are used to support cicatrization or cutaneous repair in all cases of skin damage (wound, burn, laser, abrasion, peeling) in substitution of local antiseptics and in combination with cicatrizing molecules: vitamin B5, zinc oxide paste, asiatic acid, madecassic acid, ursolic acid, avocado furans, lupine peptides, pro-cicatrization cytokine stimulants, TGF-β and IL-4.

Within the context of the invention, the expression “C7 sugar” means sugars whose sugar motif is comprised of 7 carbon atoms. The C7 sugar is advantageously selected from the group comprised of mannoheptulose, perseitol and mixtures thereof.

The source of D-mannoheptulose and/or perseitol can be a water soluble extract of sugars from avocado or from another plant. Alternately, D-mannoheptulose and perseitol are available commercially (of synthetic origin). According to an advantageous embodiment of the invention, the source of D-mannoheptulose and/or perseitol is a water soluble extract of avocado sugars.

The water soluble extract of avocado sugars can be obtained directly from any part of the avocado or avocado tree, such as the fruit, skin or pit of the avocado or the leaves or roots of the avocado tree. It is also possible to obtain a peptide extract of avocado from by-products of the avocado processing industry, including but in no way exhaustively: fresh avocado pulp, frozen pulp, dehydrated pulp, avocado oil cakes arising from oil extraction processes (mechanical extraction and/or by solvent using fruit dehydrated beforehand), de-oiled solid matter arising from wet oil extraction processes (centrifugation), de-oiled solid matter arising from enzymatic avocado oil extraction processes, crude mashed avocado (guacamole) and solid waste from units that manufacture such mashed avocado. The extract is advantageously obtained from the fresh fruit of the avocado tree. The fruits can be selected among the Hass, Fuerte, Ettinger, Bacon, Nabal, Anaheim, Lula, Reed, Zutano, Queen, Criola Selva, Mexicana Canta, Region Dschang, Hall, Booth, Peterson, and Collinson Red varieties, more advantageously among the Hass, Fuerte and Reed varieties. Preferably, the Hass, Fuerte, Ettinger and Bacon varieties will be selected, and more advantageously the Hass and Fuerte varieties.

The fruit of the avocado tree is primarily composed of water, pulp, oil and pit. The proportions of these components are, like all natural and plant matter, highly variable. However, the mean composition data presented in table 1 below, expressed in percentages of fresh fruit, are generally accepted:

TABLE 1

| | |
|---|---|
| Water | 70-85% |
| Proteins | 1.5-4.5% |
| Fats | 12-23% |
| Sugars | 1.5-5% |
| Fibers | 1.1-1.6% |

In fact, avocado is not particularly rich in polysaccharides. However, the nature of soluble monosaccharides is quite particular, such that perseitol and D-mannoheptulose are comprised of 7 carbon atoms.

The water soluble extract of avocado sugars can be obtained by a method comprising the following successive steps:
obtaining an avocado oil cake, advantageously of the fruit of avocado, by drying the avocado and then extracting the fats (oil); then
complete delipidation of said oil cake, then washing in water or in a hydroalcoholic medium, then decantation and centrifugation in order to recover a soluble fraction rich in C7 sugars (elimination of the cake);
demineralization on ionic resin of said soluble fraction obtained in the preceding step; then
ultrafiltration at 10,000 daltons; and
as needed, vacuum concentration and conditioning.

The first step of the method consists of drying the fruit and then de-oiling it. Thus, after cutting the fruit into thin slices, it can be dried by any of the techniques known to the person skilled in the art, including drying with hot air, lyophilization or osmotic drying. Generally, the temperature during this drying step, regardless of the technique used, advantageously will be maintained lower than or equal to 80° C. Within the scope of the present method, for reasons of ease of implementation and cost, drying in ventilated driers, in a thin layer and under a flow of hot air, at a temperature between 70° C. and 75° C. is preferred. The duration of the operation can vary from to 72 hours.

The fats of the dried fruit are then extracted either mechanically in a continuous screw press, or chemically using a solvent such as hexane in a soxhlet extractor or a De Smet® continuous extractor, notably according to the method described in application FR 2,843,027, or by a method using supercritical CO₂. Among the major advantages of the method, the oil co-produced constitutes a product quite obviously directly recyclable. This is why mechanical extraction of fats is preferred. The dry, de-oiled fruit, still called oil cake, can then undergo the following steps:
complete delipidation, notably in acetone and/or ethanol,
decanting and then washing of the oil cake in water and/or a hydroalcoholic mixture,
centrifugation, filtration, recovery of the soluble fraction (elimination of the cake),
concentration,
demineralization by ion exchange,
ultrafiltration with a 10 kDa cutoff,
vacuum concentration, addition of preservative and conditioning.

Generally, the final aqueous extract can contain 0.1% to 20% dry matter by weight, advantageously 1% to 10% dry matter, even more advantageously 3% to 5% dry matter. The content in C7 sugars, i.e., in D-mannoheptulose and perseitol, in the dry matter is advantageously between 50% and 100% by weight, more particularly between 65% and 90% by weight, compared to the total weight of dry matter. The mean analytical data for an aqueous solution with 5% dry extract, obtained by the method described above, are given in following table 2:

TABLE 2

| pH (¼ dilution) | | 3-5 |
|---|---|---|
| Absorbance | 420 nm | Less than 0.200 |
| (½ dilution) | 550 nm | Less than 0.050 |

| | |
|---|---|
| C7 sugars/dry matter | 50-100% |

The relative composition in sugars of the water soluble extract, by weight compared to the total weight of dry matter of the extract, advantageously meets the following criteria (relative composition determined by high performance liquid chromatography (HPLC)):

| | |
|---|---|
| D-mannoheptulose | 0-100%, in particular 5-80%, |
| Perseitol | 0-100%, in particular 5-80%, |
| Sucrose | less than 10%, |
| Glucose | less than 10%, |
| Fructose | less than 10%. |

The water soluble extract of avocado sugars advantageously comprises, compared to the total weight of dry matter, 10% to 80% by weight of D-mannoheptulose, more advantageously 15% to 70% by weight of D-mannoheptulose. The water soluble extract of avocado sugars advantageously comprises, compared to the total weight of dry matter, 20% to 80% by weight of perseitol, more advantageously 25% to 70% by weight of perseitol.

Preferably, the relative sugar composition of the water soluble extract, by weight compared to the total weight of dry matter of the extract, meets the following criteria (relative composition determined by HPLC):

| | |
|---|---|
| D-mannoheptulose | 25% to 60%, |
| Perseitol | 25% to 60%, |
| Sucrose | less than 10%, |
| Glucose | less than 10%, |
| Fructose | less than 10%. |

Surprisingly, the inventors noted a synergistic effect between D-mannoheptulose and/or perseitol and the minority sugars (fructose, glucose, sucrose) present in the extract of avocado sugars.

The extract obtained optionally can be lyophilized for the purpose of obtaining a solid powder (dry extract) that is completely water soluble.

The extract obtained optionally can be fractioned into each purified sugar. This separation and purification can be carried out by any technique known to the person skilled in the art, including but in no way exhaustively precipitation and filtration, recrystallization, or separation by chromatography such as the improved simulated moving bed (ISMB) method.

Within the scope of the present invention, the composition can further comprise (with or without the compounds listed above) organic acids, sugars that can and cannot be metabolized by the body, excipients such as polyols, maltodextrines, silica derivatives, titanium oxides, glycols such as pentylene glycol or butylene glycol, caprylyl glycol or active ingredients to maintain cutaneous flora.

Within the scope of the present invention, the composition can further comprise (with or without the compounds listed above) at least one compound selected from the group comprised of glycols, hydrating active ingredients, keratin synthesis activators, keratin regulators, keratolytics, agents that restructure the cutaneous barrier (cutaneous lipid synthesis activators, peroxisome proliferator activated receptor (PPAR) agonists), keratinocyte differentiation activators (retinoids, Calcidone®, calcium, vitamin D3, vitamin C), keratinocyte proliferation activators (retinoids and vitamin B5), antibiotics, antibacterials, antifungals (potassium sorbate, parabens, piroctone olamine, zinc pyrithione, cyclopyrox, selenium salts, desquamation regulators-coal tar and ichtyol pale), antivirals, seboregulators, such as 5-a reductase inhibitors, notably the active ingredient 5-alpha Avocuta® marketed by Expanscience Laboratories, immunomodulators such as tacrolimus and pimecrolimus, oxazolines, preservatives, anti-irritants (thermal water and oat derivatives), soothing agents, sun filters and screens, antioxidants, growth factors, cicatrizants or eutrophic molecules, anti-inflammatories and compounds containing vegetable oil unsaponifiables.

The keratin synthesis activators that can be used within the scope of the present invention in combination with C7 sugars, advantageously D-mannoheptulose and/or perseitol, are advantageously retinoids, lupine peptides (marketed by Silab), key proteins of the stratum corneum or granulosum (keratins).

The antibiotics that can be used within the scope of the present invention in combination with C7 sugars, advantageously D-mannoheptulose and/or perseitol, are advantageously fusidic acid, penicillin, tetracyclines, pristinamycin, erythromycin, clindamycin, mupirocin, minocycline and doxycycline. The antivirals that can be used within the scope of the present invention in combination with C7 sugars, advantageously D-mannoheptulose and/or perseitol, are advantageously acyclovir and valacyclovir. The anti-irritants that can be used within the scope of the present invention in combination with C7 sugars, advantageously D-mannoheptulose and/or perseitol, are advantageously glycine, lupine sugars and/or peptides and Cycloceramide® (oxazoline derivative).

The soothing agents that can be used within the scope of the present invention in combination with C7 sugars, advantageously D-mannoheptulose and/or perseitol, are advantageously alpha bisabolol and liquorice derivatives. The keratin regulators that can be used within the scope of the present invention in combination with C7 sugars, advantageously D-mannoheptulose and/or perseitol, are advantageously alpha-hydroxy acids and derivatives thereof. The keratolytics that can be used within the scope of the present invention in association with C7 sugars, advantageously D-mannoheptulose and/or perseitol, are notably salicylic acid and derivatives thereof. The antioxidants that can be used within the scope of the present invention in combination with C7 sugars, advantageously D-mannoheptulose and/or perseitol, are advantageously vitamins (C, E) and trace elements (copper, zinc, selenium). The growth factors that can be used within the scope of the present invention in combination with C7 sugars, advantageously D-mannoheptulose and/or perseitol, are advantageously becaplermin and TGF-β (transforming growth factor beta).

The cicatrizants that can be used within the scope of the present invention in combination with C7 sugars, advantageously D-mannoheptulose and/or perseitol, are advantageously vitamin A, panthenol, Avocadofurane®, zinc oxide, magnesium, silicon, madecassic acid or asiatic acid.

The drugs that can be used within the scope of the present invention in combination with C7 sugars, advantageously D-mannoheptulose and/or perseitol, are advantageously drugs suited for topical or oral administration to prevent and/or treat atopy (corticoids, emollients), acne (antibiotics, benzoyl peroxide, retinoids, azelaic acid, vitamin PP, zinc, cyclins), eczema (immunomodulators, emollients, salmon oil, borage, prebiotics, probiotics) or psoriasis (corticoids, calcipotriol, calcitriol, tazarotene, cade oil, acitretine, PUVA therapy). The anti-inflammatories that can be used within the scope of the present invention in combination with D-mannoheptulose and/or perseitol are advantageously steroidal anti-inflammatory drugs (SAIDs) such as corticoids, or non-steroidal anti-inflammatory drugs (NSAIDs).

The agents that restructure the cutaneous barrier and help stimulate synthesis of key epidermal lipids and that can be used within the scope of the present invention in combination, advantageously with a synergistic effect, with C7 sugars, advantageously D-mannoheptulose and/or perseitol, are advantageously sunflower concentrates, even more advantageously linoleic sunflower concentrates, such as the active ingredient marketed by Expanscience Laboratories, Soline® (see international application WO 01/21150), vegetable oil unsaponifiables, such as Avocadofurane® (see international application WO 01/21150), and PPAR agonists (rosiglitazone, pioglitazone). The restructuring agents are advantageously present in a proportion ranging from 0.001% to 30% by weight, compared to the total weight of the composition or drug. The antifungals that can be used within the scope of the present invention in combination with C7 sugars, advantageously D-mannoheptulose and/or perseitol, are advantageously econazole and ketoconazole.

The antiseptic preservatives that can be used within the scope of the present invention in combination with C7 sugars, advantageously D-mannoheptulose and/or perseitol, are for example triclosan, chlorhexidine and quaternary ammoniums. The immunomodulators that can be used within the scope of the present invention in combination, advantageously with a synergistic effect, with C7 sugars, advantageously D-mannoheptulose and/or perseitol, are advantageously tacrolimus, pimecrolimus and oxazolines.

The oxazolines that can be used within the scope of the present invention in combination, advantageously with a synergistic effect, with C7 sugars, advantageously D-mannoheptulose and/or perseitol, are advantageously oxazolines selected from the group comprised of 2-undecyl-4-hydroxymethyl-4-methyl-1,3-oxazoline, 2-undecyl-4,4-dimethyl-1,3-oxazoline, (E)-4,4-dimethyl-2-heptadec-8-enyl-1,3-oxazoline, 4-hydroxymethyl-4-methyl-2-heptadecyl-1,3-oxazoline, (E)-4-hydroxymethyl-4-methyl-2-heptadec-8-enyl-1,3-oxazoline and 2-undecyl-4-ethyl-4-hydroxymethyl-1,3-oxazoline. Even more advantageously, said oxazoline is 2-undecyl-4,4-dimethyl-1,3-oxazoline, called OX-100 or Cycloceramide®.

The compounds containing vegetable oil unsaponifiables that can be used within the scope of the present invention in combination, advantageously with a synergistic effect, with C7 sugars, advantageously D-mannoheptulose and/or perseitol, are advantageously selected from the group comprised of avocado furan lipids, unsaponifiables of avocado and soy, lupine oil concentrates, sunflower oil concentrates and mixtures thereof.

The avocado furan lipids that can be used within the scope of the present invention in combination, advantageously with a synergistic effect, with C7 sugars, advantageously D-mannoheptulose and/or perseitol, are advantageously natural 2-alkyl furans, notably the active ingredient Avocadofurane® marketed by Expanscience Laboratories, which can be obtained by the method described in international application WO 01/21605.

The unsaponifiables of avocado and soy that can be used within the scope of the present invention in combination, advantageously with a synergistic effect, with C7 sugars, advantageously D-mannoheptulose and/or perseitol, are advantageously a mixture of furan unsaponifiables of avocado and unsaponifiables of soy in a ratio of approximately ⅓-⅔, respectively. The unsaponifiables of avocado and soy are even more advantageously the product Piascledine®, marketed by Expanscience Laboratories.

The lupine oil concentrates that can be used within the scope of the present invention in combination, advantageously with a synergistic effect, with C7 sugars, advantageously D-mannoheptulose and/or perseitol, are advantageously concentrates obtained by molecular distillation of lupine oil, advantageously sweet white lupine oil, such as those described in international application WO 98/47479. They advantageously contain approximately 60% unsaponifiables by weight.

The sunflower oil concentrates that can be used within the scope of the present invention in combination, advantageously with a synergistic effect, with C7 sugars, advantageously D-mannoheptulose and/or perseitol, are advantageously linoleic sunflower concentrates, such as the active ingredient marketed by Expanscience Laboratories, Soline® (see international application WO 01/21150).

According to an advantageous embodiment of the invention, the inventive composition further comprises at least one agent selected from the group comprised of antifungals (standard pharmaceuticals or cosmetics, such as defined above), antibacterials (such as those defined above), antibiotics (standard or endogenous, such as those defined above), antivirals (such as those defined above), lipid plant extracts containing unsaponifiables such as sunflower concentrates, avocado concentrates and soy concentrates (such as those defined above), zinc oxide, vitamins (such as those defined above), plant peptides and sugars (such as those defined above), anti-inflammatories (such as those defined above), keratinocyte differentiation and/or proliferation activators (such as those defined above).

The composition (i.e., the end product to be marketed) advantageously comprises 0.001% to 20% by weight, more advantageously 0.001% to 10% by weight, even more advantageously 0.01% to 10% by weight, even more advantageously 0.01% to 5% by weight, even more advantageously 0.01% to 2% by weight of C7 sugars, compared to the total weight of the composition. The C7 sugars are preferably mannoheptulose, perseitol and mixtures thereof.

According to an advantageous embodiment of the invention, the composition advantageously comprises 0.01% to 20% by weight, more advantageously 0.01% to 10% by weight, even more advantageously 0.05% to 5% by weight, even more advantageously 0.05% to 2% by weight of an extract of avocado sugars such as defined above, compared to the total weight of the composition.

Optimal modes of administration, dosing schedules and galenic forms of the composition can be determined according to criteria generally taken into account in establishing a pharmaceutical, dermatological, cosmetological or veterinary treatment via local or systemic route.

The treatment can be particularly suitable for immunosuppressed or sensitive individuals (pregnant women, overweight or obese individuals, diabetics) as well as for individuals resistant to antifungals or those who seek to avoid becoming resistant by limiting the content of and contact with the azole molecules typically used.

### EXAMPLE 1

### Preparation of a Water Soluble Extract of Avocado Sugars

Fifty kilograms of fresh avocados, the Hass variety, are cut into slices 2-5 mm thick, pit included, using a slicer with a circular blade. The drying apparatus is a temperature controlled hot air drying oven. The sliced avocados are distributed in a thickness of 4-5 cm on stacked racks. Drying is done for 48 hours at a temperature of 80° C. Once dried, the fruits are subjected to cold pressing. This operation is carried out on a small Komet® laboratory press. Four kilograms of de-fatted dry fruit (oil cakes) are thus obtained.

The de-fatted fruits are cold crushed and then extracted at reflux in the presence of 25 liters of ethanol. The fat-depleted powder is then recovered by filtration on a Büchner funnel and oven dried at 50° C. for 5 hours.

The oil cake is then washed with a 70/30 hydroalcoholic mixture (10 liters) and then separated by centrifugation. The soluble fraction (liquid) is recovered to be purified and concentrated according to the following procedure:
Demineralization using ionic resins: demineralization of C7 sugars by passing through OH⁻ resins and then H⁺ resin.
10,000 Da ultrafiltration: ultrafiltration is carried out with a system equipped with four 10 kDa cutoff membranes.
Vacuum concentration: concentration of the purified extract is carried out using a vacuum evaporator until roughly 6% dry matter is obtained.
Conditioning: the concentration of the extract is adjusted to 5% dry matter, and then the extract undergoes sterilizing filtration with a 0.2 μm cutoff membrane.

Following table 3 gives the composition of the extract of avocado C7 sugars, at 5% dry matter, prepared according to the method described above:

TABLE 3

| Appearance | Light yellow |
|---|---|
| | solution |
| Analytical criteria | |
| Dry matter | 5% |
| pH | 4.0 |
| Absorbance at 420 mm (½ dilution) | 0.008 |
| Absorbance at 550 mm (½ dilution) | 0.004 |
| Composition (% of dry matter) | |
| Sucrose | 1.8 |
| Glucose | 3.0 |
| D-mannoheptulose | 38.6 |
| Fructose | 4.4 |
| Perseitol | 38.0 |

According to this same method, another extract was prepared, whose pH, absorbance and C7 sugar content are given in table 4 below. The content in C7 sugar corresponds to the sum of perseitol and D-mannoheptulose analyzed by HPLC.

TABLE 4

| Dry matter | | 5% |
|---|---|---|
| pH | | 4.0 |
| Absorbance | 420 nm | 0.010 |
| (½ dilution) | 550 nm | 0.005 |

| | |
|---|---|
| C7 sugars/dry matter | 83.6 |

### EXAMPLE 2

### Effects of the Extract of Avocado Sugars on Yeast Pathogenicity

The effects of the extract of avocado sugars described in example 1 (AV119) were analyzed on the capacities of invasion of yeasts including Malassezia furfur (a dimorphic, lipid-dependant yeast that is a commensal of normal human cutaneous flora) and Candida albicans. The effect of these yeasts and AV119 on the expression of proinflammatory and immunomodulatory keratinocyte cytokines was also analyzed.

1. Microorganisms Used

Staphylococcus aureus ATCC 29737, Streptococcus pyogenes ATCC 10389, Pseudomonas aeruginosa ATCC 25619, Candida albicans ATCC 53324 and Malassezia furfur ATCC 12078 are obtained from the American Type Culture Collection (Rockville, Md., USA).

The bacterial strains are used as a negative control and the yeast C. albicans is used as a positive control. The principal target of the study is Malassezia furfur.

2. Cell Culture and Treatment

The tests are carried out on normal human keratinocytes obtained from surgical samples of normal adult skin.

3. Study of the Effect of AV119 on Microorganism Viability and Growth

In order to study the effects of AV119 on the viability of S. aureus, P. aeruginosa, S. pyogenes, C. albicans and M. furfur, various concentrations of AV119 are used (0.03% to 0.5% dry matter) to determine if there is inhibition of bacterial and yeast growth.

Antimicrobial activity is determined by the modified agar gel diffusion method of Perez et al. (Perez C. et al. An antibiotic assay by the agar well diffusion method. Acta Biologiae et Medicine Experimentalis. 1990; 15: 113-115).

The plates are incubated at 35±2° C. for 24 hours, except for C. albicans (incubation at 30° C.) and M. furfur (incubation at 30° C. for 4 days). Measurement of the diameter of the inhibition zone establishes the antimicrobial effect.

Living microorganisms were determined after 24 h of AV119-microorganism contact by counting the colony forming units (CFUs) on the selective agar medium in order to exclude a direct antimicrobial effect of AV119.

The results obtained show that the microorganisms analyzed (S. aureus, P. aeruginosa, S. pyogenes, M. furfur and C. albicans) are all resistant to AV119. Indeed, at all concentrations tested, no significant effect on the viability or growth of the bacteria and yeasts is observed.

These results exclude a direct bactericidal effect of AV119.

4. Tests of Invasion by S. aureus, P. aeruginosa and S. pyogenes

The keratinocytes are pretreated with 0.1% AV119 for 24 hours, and then are infected with suspensions of bacteria (100 bacteria per cell) for 2 hours at 37° C.

The cells are then washed, incubated for 2 hours at 37° C. and then lysed. The cellular lysates are then diluted in PBS and deposited on a selective agar medium in order to quantify the number of living intracellular bacteria.

AV119 does not modify the capacities of invasion of S. aureus, P. aeruginosa and S. pyogenes.

5. Tests of Invasion by M. furfur and C. albicans

Human keratinocytes are pretreated for 24 hours with 0.1% AV119 and then are infected by M. furfur (yeast/keratinocyte:30/1) or by C. albicans (yeast/keratinocyte:10/1) for 24, 48 or 72 hours.

Next, the cells are washed, stained (May-Grunwald-Giemsa) and observed under the microscope.

Percentage of incorporation is evaluated by counting the number of keratinocytes in which M. furfur or C. albicans penetrated.

The results show that the sugars inhibit the absorption of M. furfur and C. albicans (table 5).

Indeed, in the keratinocytes treated with AV119, yeast penetration is not observed (penetration observed in the untreated keratinocytes).

TABLE 5
Effect of AV119 on the capacity of invasion
of Malassezia furfur and Candida albicans

| | Number of yeasts internalized | | | |
|---|---|---|---|---|
| | (mean ± SD) | | | |
| | M. furfur | | C. albicans | |
| Growth conditions | 48 h | 72 h | 48 h | 72 h |
| Keratinocyte-SFM | 155 ± 18 | 148 ± 16 | 225 ± 23 | 247 ± 31 |
| medium | | | | |
| Keratinocyte-SFM | 150 ± 21 | 153 ± 15 | 195 ± 18 | 205 ± 27 |
| medium + | | | | |
| 0.025% AV119 | | | | |
| Keratinocyte-SFM | 140 ± 19 | 137 ± 13 | 203 ± 13 | 197 ± 31 |
| medium + | | | | |
| 0.050% AV119 | | | | |
| Keratinocyte-SFM | 14 ± 7 | 16 ± 4 | 36 ± 11 | 32 ± 5 |
| medium + | | | | |
| 0.1% AV119 | | | | |
| Keratinocyte-SFM | 15 ± 4 | 19 ± 3 | 41 ± 6 | 37 ± 3 |
| medium + | | | | |
| 0.2% AV119 | | | | |

6. Flocculation Tests

Flocculation is quantified by measuring the optical density (at 660 nm) of a suspension of M. furfur and C. albicans after agitation and a subsequent rest period in order for the flocculates to sediment.

The tests are carried out in the presence of 0.1% AV119 or glucose (negative control) or mannan (positive control).

The flocculation tests are carried out in the presence of various concentrations of CaCl₂, given that Ca²⁺ ions are necessary for flocculation.

The tubes are agitated for 15 minutes and left to rest for 15 min after which optical density at 660 nm is measured.

An aggregation is observed; the yeast cells form large aggregates.

TABLE 6
Flocculation results (OD660) for C. albicans

| Log [Ca⁺⁺] | −1 | 0 | 1 | 2 |
|---|---|---|---|---|
| C. albicans | 1.95 | 1.97 | 1.82 | 1.91 |
| C. albicans + AV119 | 1.93 | 1.2 | 0.7 | 0.6 |

TABLE 7
Flocculation results (OD660) for M. furfur

| Log [Ca⁺⁺] | −1 | 0 | 1 | 2 |
|---|---|---|---|---|
| M. furfur | 2 | 2.1 | 1.98 | 1.99 |
| M. furfur + AV119 | 1.98 | 1.1 | 0.5 | 0.5 |

It is thus noted that in the absence of AV119 the yeasts C. albicans and M. furfur do not aggregate. Adding AV119 causes flocculation of these two yeasts.

7. Analysis of the Expression of Proinflammatory Cytokines by Real Time RT-PCR

To determine if AV119 interferes with the mechanism of action of M. furfur, the expression of several cytokines known to be controlled by yeast is analyzed.

Expression of the mRNA of IL-1α, IL-6, IL-8 and TGF-β1 in human keratinocytes is determined by real time RT-PCR.

The cells are pretreated with 0.1% AV119 for 24 hours and then treated with M. furfur (yeast/keratinocyte:30/1) for 24 and 48 hours.

It is known that M. furfur acts most notably by decreasing the expression of the cytokine IL-1α and by increasing the production of TGF-β1. Thus, yeast modulates the proinflammatory response of the host to allow its survival.

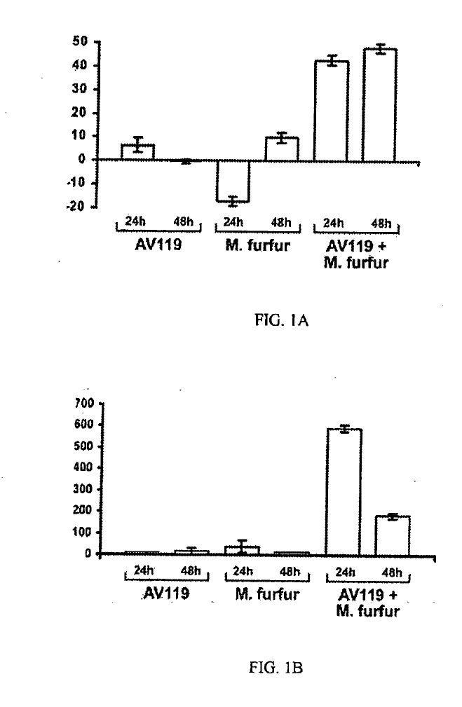

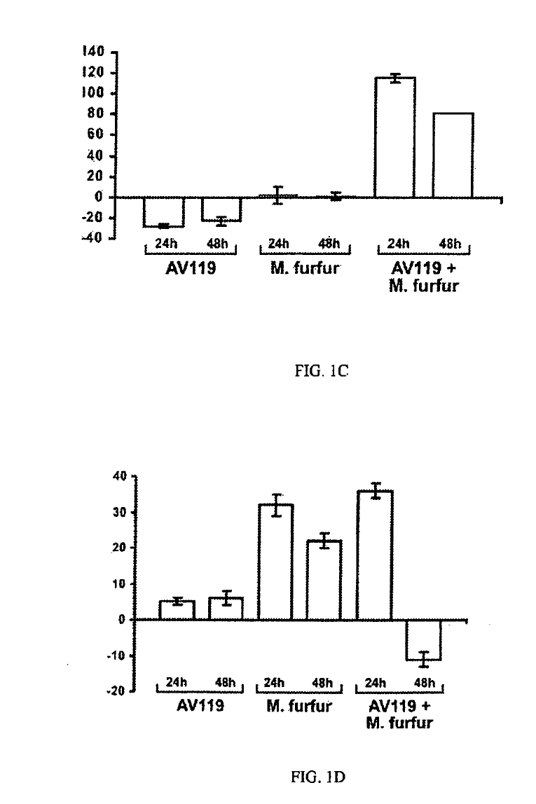

The results (FIGS. 1A to 1D) show that AV119 enables keratinocytes to restore their capacity to respond to invasion by yeast with a proper proinflammatory reaction.

### LEGEND OF FIGURES

FIG. 1A: increase in the level of IL-1α (% compared to control) for, from left to right:
AV119, 24 hours and 48 hours
M. furfur, 24 hours and 48 hours
AV119+M. furfur, 24 hours and 48 hours

FIG. 1B: increase in the level of IL-6 (% compared to control) for, from left to right:
AV119, 24 hours and 48 hours
M. furfur, 24 hours and 48 hours
AV119+M. furfur, 24 hours and 48 hours

FIG. 1C: increase in the level of IL-8 (% compared to control) for, from left to right:
AV119, 24 hours and 48 hours
M. furfur, 24 hours and 48 hours
AV119+M. furfur, 24 hours and 48 hours

FIG. 1D: increase in the level of TGF-β (% compared to control) for, from left to right:
AV119, 24 hours and 48 hours
M. furfur, 24 hours and 48 hours
AV119+M. furfur, 24 hours and 48 hours

Treating keratinocytes (infected by M. furfur) with AV119 leads to an increase in the production of cytokines IL-1α, IL-6 and IL-8 compared to untreated keratinocytes infected by M. furfur. This increase reaches 43% for IL-1α (FIG. 1A) after 24 hours of treatment, whereas for IL-6 and IL-8 it reaches more than 100% (FIGS. 1B and 1C). Conversely, a significant decrease in the production of TGF-β1 is observed after 48 hours in response to treatment with AV119 (FIG. 1D).

These results show that the defensive capacity of the keratinocytes is restored.

Finally, the possible salting out of M. furfur metabolites in the supernatants of the treated keratinocytes was investigated. Keratinocytes treated with AV119 or untreated are incubated with the supernatant obtained from cells treated with AV119 and infected by M. furfur. The response of the keratinocytes is not changed by the presence or absence of the supernatant, indicating the absence of a specific metabolite able to induce a proinflammatory response in the cells treated with AV119 and infected by M. furfur.

Note: Statistical Analysis

Each experiment is repeated at least 5 times.

The results are expressed in the form of the mean the standard deviation (SD).

ANOVA tests are carried out for each experiment. The value of p is generally evaluated between 0.01 and 0.03, which confirms the statistical relevance of the results obtained (p<0.05).

1.-9. (canceled)

10. A method for preventing and for treating with long term efficacy mycoses selected from the group comprised of candidiases and pityrosporoses, comprising the administration to a person in need thereof of a composition comprising at least one C7 sugar.

11. The method according to claim 10, wherein the candidiases are selected from the group comprised of candidal intertrigo, oral-digestive candidiasis, genital candidiasis, candidal onychitis and perionychitis, chronic mucocutaneous candidiasis and candidal folliculitis.

12. The method according to claim 11 wherein the candidiases are selected from the group comprised of oral-digestive candidiasis, genital candidiasis, infantile candidiasis of the genitals and buttocks, in particular diaper rash, and candidal folliculitis.

13. The method according to claim 12 wherein the candidiase is diaper rash.

14. The method according to claim 10, wherein pityrosporoses are selected from the group comprised of pityriasis versicolor, pityriosporum folliculitis, acne, rosacea, atopic dermatitis and seborrheic dermatitis.

15. The method according to claim 14 wherein the pityrosporoses are selected from the group comprised of pityriosporum folliculitis, seborrheie dermatitis, acne, rosacea and atopic dermatitis.

16. The method according to claim 10, wherein the C7 sugar is selected from the group comprised of mannoheptulose, perseitol and mixtures thereof.

17. The method according to claim 10, wherein the source of the C7 sugars is a water soluble extract of avocado sugars.

18. The method according to claim 17, wherein the water soluble extract of avocado sugar is obtained by a method comprising the following successive steps:
obtaining an avocado oil cake, by drying the avocado and then extracting the fats; then
cryogrinding, complete delipidation and hydroalcoholic or aqueous extraction of said oil cake, then decantation and centrifugation in order to recover a soluble fraction rich in C7 sugars (elimination of the cake);
demineralization on ionic resin of said soluble fraction obtained in the preceding step; then
ultrafiltration at 10,000 daltons; and
vacuum concentration and conditioning.

19. The method according to claim 18 wherein the avocado oil cake is obtained from the fruit of avocado.

20. The method according to claim 10, wherein the source of C7 sugars is a water soluble extract of avocado sugars which comprises by weight, compared to the total weight of dry matter of the extract (relative composition determined by HPLC):

| | |
|---|---|
| D-mannoheptulose | 1% to 90% |
| Perseitol | 1% to 90% |
| Sucrose | less than 10% |
| Glucose | less than 10% |
| Fructose | less than 10% |

21. The method according to claim 10, wherein the composition comprises 0.001%. to 20% by weight of C7 sugar, compared to the total weight of the composition.

22. The method according to claim 21, wherein the C7 sugar is selected from the group comprised of mannoheptulose, perseitol and mixtures thereof.

23. The method according to claim 10, wherein the composition comprises 0.01% to 20% by weight of said water soluble extract of avocado sugars, compared to the total weight of the composition.

24. The method according to claim 10, wherein the composition further comprises at least one active ingredient selected from the group comprised of antifungals, antibacterials, antibiotics, antivirals, lipid vegetable extracts containing unsaponifiables, zinc oxide, vitamins, plant peptides and sugars, anti-inflammatories and keratinocyte differentiation and/or proliferation activators.

25. The method according to claim 24 wherein the lipid vegetable extracts is selected from the group comprised of sunflower concentrates, avocado concentrates, soy concentrates and mixtures thereof.
